(19) United States
(12) Patent Application Publication
(10) Pub. No.: US 2026/0114771 A1
(43) Pub. Date: Apr. 30, 2026

(54) METHOD AND SYSTEM FOR BIOMARKER DETECTION OF ACUTE KIDNEY INJURY (AKI)

(71) Applicant: Blue Ridge Biomedical LLC, Hanahan, SC (US)

(72) Inventors: Calvin A. Chernyatinskiy, Fort Mill, SC (US); Omar M.H. Aguilar, JR., Hanahan, SC (US); Aaron E. Spearman, Travelers Rest, SC (US); Nicholas P. Stiebler, Charleston, SC (US); Alexander R. Bowie, LaGrange, GA (US)

(21) Appl. No.: 19/368,503

(22) Filed: Oct. 24, 2025

Related U.S. Application Data
(60) Provisional application No. 63/712,231, filed on Oct. 25, 2024.

(51) Int. Cl. A61B 5/20; A61B 5/00; A61B 5/145; A61B 5/1486; A61B 10/00; G01N 27/12

(52) U.S. Cl. CPC A61B 5/201; A61B 5/14507; A61B 5/1486; A61B 5/207; A61B 5/6852; A61B 10/007; G01N 27/12

(57) ABSTRACT

A device for detecting an analyte is described. The device comprises a sample chamber comprising an inlet port, a testing chamber and an electrochemical sensor, wherein the electrochemical sensor is in electrical communication with a working electrode that specifically binds at least one biomarker, and wherein upon binding an output of the working electrode is changed. Additionally, disclosed herein is a system comprising a device and a catheter detachably coupled to the device. Furthermore, a method for detecting kidney injury in a patient is described.

### CROSS REFERENCE TO RELATED APPLICATION

This application claims the filing benefit of U.S. Provisional Patent Application Ser. No. 63/712,231 having a filing date of Oct. 25, 2024, which is incorporated herein by reference for all purposes.

### BACKGROUND

The causes of acute kidney injury (AKI) can be classified into pre-renal, post-renal, and intrinsic AKI. Pre-renal AKI is caused by hypoperfusion (ischemia) to the kidneys, post-renal by the obstruction of urine flow, and intrinsic AKI by structural damage to the kidneys. Current clinical diagnostic methods are based on increased serum creatinine levels with or without a decrease in urine output.

AKI has a high prevalence in hospitals, complicating roughly 15% of inpatient admissions. Among critically ill patients, the prevalence of AKI is even higher, impacting 30-50% of ICU patients. AKI is also associated with substantial patient mortality. The estimated in-hospital mortality rate stands at 20-25%. This figure again increases in critically ill patients, where individuals needing renal replacement therapy due to AKI experience mortality rates exceeding 50%.

### SUMMARY

The present disclosure is generally directed to a device for detecting an analyte, the device comprising a sample chamber comprising an inlet port, a testing chamber and an electrochemical sensor, wherein the electrochemical sensor is in electrical communication with a working electrode that specifically binds at least one biomarker, and wherein upon binding, an output of the working electrode is changed.

In embodiments of the present disclosure, a system comprising a device and a catheter detachably coupled to the device is disclosed. The device may comprise a sample chamber comprising an inlet port, a testing chamber and an electrochemical sensor, wherein the electrochemical sensor is in electrical communication with a working electrode that specifically binds at least one biomarker, and wherein upon binding an output of the working electrode is changed.

In embodiments of the present disclosure, a method for detecting kidney injury in a patient is described, the method comprising collecting a urine sample comprising a biomarker from the patient, wherein the urine is collected through a catheter in fluidic communication with a device, wherein the device comprises: a sample chamber fluidically connected to the catheter, a testing chamber fluidically connected to the sample chamber and an electrochemical sensor comprising a microprocessor in electrical communication with a working electrode on an interior surface of the testing chamber. The method further comprises passing the urine from the catheter to the testing chamber and determining the concentration of the biomarker in the urine.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is an isometric view of a device of the present disclosure;

FIG. 2A is a simplified drawing of the device of FIG. 1;

FIG. 2B is a simplified drawing of the device of FIG. 1;

FIG. 2C is a simplified drawing of the device of FIG. 1;

FIG. 2D is a simplified drawing of the device of FIG. 1;

FIG. 3A is a simplified drawing of the device of FIG. 1;

FIG. 3B is a simplified drawing of the device of FIG. 1;

FIG. 3C is a simplified drawing of the device of FIG. 1;

FIG. 3D is a simplified drawing of the device of FIG. 1;

FIG. 4 is a view of the device when used in the management of a patient;

FIG. 5 is a flowchart for diagnosing AKI using the device of the present disclosure;

FIG. 6 is a flowchart showing a valve-control sequence; and

FIG. 7 is a graph showing the concentration of the biomarker NGAL increasing above a threshold in the time following heart surgery.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

One method used in medical settings to detect kidney injury is measuring the creatinine concentration in blood serum. Creatinine is a waste product that is typically filtered out by healthy kidneys. Therefore, an elevated concentration of creatinine in a patient's blood may be indicative of AKI. However, the issues with said method of detecting AKI are several. As a first matter, the baseline serum concentration of creatinine varies throughout patient populations and across individuals. Therefore, the determination of an “elevated” serum creatinine is a relative one. Further, serum creatinine concentration is non-specific to AKI—there are a variety of other conditions that may be responsible for elevated serum creatinine concentration. Additionally, laboratory tests for serum creatinine levels, even in rushed settings, typically require thirty minutes to one hour to complete. Measurement of serum creatinine additionally requires collecting a blood sample from a patient, such as through a blood draw. In some patients, a blood draw may be difficult and/or deleterious to a patient's health.

Described herein is a system and method that makes use of a device to rapidly assess a patient's AKI status and future likelihood of AKI by assessing the concentration of a biomarker in a fluid, such as urine. Beneficially, the device of the present disclosure may be able to rapidly assess as patient's AKI status by utilizing an already in-place catheter. Thus, the device of the present disclosure obviates the difficulty presented by current methods, e.g., requiring a blood draw to measure serum creatinine.

The device of the present disclosure comprises a frame, a sample chamber, a testing chamber and an electrochemical sensor disposed therein. The device may be incorporated into catheter systems, or coupled to a pre-existing catheter so as to be detachable. For instance, a patient may have an existing catheter, such as a Foley catheter. The device may be able to connect to the pre-placed catheter. The catheter and device may be combined to form a sterile, closed loop assembly for urine collection and testing.

The device may comprise an inlet port, whereby fluid may enter the device from the catheter. The inlet port is not particularly limited, but includes barbs, luer lock/slip connectors, compression fittings, push-to-connect/quick-connect fittings, clamps, magnetic couplings, snap-fit couplings and adhesives. In general, the inlet port and the catheter may be coupled together to form a seal against fluid leakage. Further, disposed between the inlet port and the sample chamber may be a filter. The filter may be of a mesh fine enough to prevent particulate, such as urinary tract debris, from entering the device. The device may further comprise a discharge port that also may comprise of the same characteristics as the inlet port, whereby fluid may exit the sample chamber and the device.

In embodiments of the present disclosure, the sample chamber and the testing chamber may be in fluidic communication by way of a sample in-flow tube disposed between the sample chamber and the testing chamber.

In embodiments of the present disclosure, the testing chamber may be in fluidic communication with the inlet port directly. For instance, the inlet port may comprise a 3/2 valve, the valve states of which may include inlet port-sample chamber and inlet port-testing chamber. In such embodiments, the testing chamber may also be in fluidic communication directly with the discharge port, such as by similar means to the inlet port previously described. In embodiments wherein the testing chamber can be in fluidic communication directly with the inlet port, the sample in-flow tube may not be required. The testing chamber may further comprise a vent. The vent may allow for pressure equalization when the testing chamber is drained.

In embodiments of the present disclosure, the inlet port, sample in-flow tube and discharge port may comprise apertures that can be closed or opened with a valve. For instance, the inlet port, sample in-flow tube and discharge port themselves may comprise valves. Alternatively, a valve exterior to the inlet port, sample in-flow tube and discharge port may actuate, thereby impinging on and restricting the flow of fluid through the port/tubes. Valves of the present disclosure may comprise gated valves, ball valves, solenoid valves, isolation valves, pinch valves, proportional valves or general-service valves.

The electrochemical sensor may comprise a microprocessor in electrical communication with a working electrode, reference electrode and counter electrode. In embodiments, the working electrode may comprise a screen printed electrode. The screen printed electrode may comprise a conductive material such as gold, carbon, graphene, etc. The working electrode may specifically bind one or more biomarkers present in the fluid at elevated concentrations in urine in cases of AKI. The biomarkers may include tissue inhibitor of metalloproteinase 2 (TIMP-2), insulin-like growth factor binding protein 7 (IGFBP7), neutrophil gelatinase-associated lipocalin (NGAL), kidney injury molecule-1 (KIM-1), Interleukin-18 (IL-18), liver fatty acid binding protein (L-FABP), cystatin C, N-acetyl-β-glucosaminidase (NAG), Glutathione S-transferase pi (GST), clusterin, calprotectin, β2-microglobulin, Trefoil Factor 3 (TFF3), vanin-1, osteopontin, cytokines, and urinary exosomes or RNA. The working electrode may be a molecularly imprinted sensor, an electrochemical aptamer sensor or an electrochemical antibody sensor. In embodiments, the working electrode may be miniaturized to reduce sample volume requirements, increase surface-area-to-volume ratio, and enhance electrochemical sensitivity. The electrode dimensions may be selected based on desired detection limits, fabrication constraints, or fluidic channel geometry. For instance, a miniaturized electrode may have a surface area of less than 2 cm², such as less than 1 cm², such as less than 0.5 cm².

The working electrode may be disposed in an interior of the testing chamber. The interior of the testing chamber may further comprise the reference electrode and the counter electrode. In embodiments, the electrochemical sensor may comprise the working, reference or counter electrodes, in combination with the microprocessor formed as an integrated component. In embodiments wherein the electrodes and the microprocessor are formed as an integrated electrochemical sensor, the integrated electrochemical sensor may be inserted into the testing chamber. The inserting of the integrated electrochemical sensor into the testing chamber may position the electrodes into the interior of the testing chamber. After insertion, the electrochemical sensor may be sealed into the testing chamber.

The sizes of the sample chamber and testing chamber are not particularly limited, but may be large enough to provide adequate room for the electrodes. For instance, the sample chamber and testing chamber may have substantially the same volume, or different volumes. While not limited to a specific volume, the sample chamber, testing chamber, or both may have an internal volume from 5 mL to 50 mL, such as from 10 mL to 35 mL. However, in versions wherein a miniature sized electrochemical sensor is used, the sample chamber, testing chamber, or both, may have an internal volume from 100 μL to 1 mL, such as from 200 μL to 800 μL.

In embodiments of the present disclosure, the electrochemical sensor may comprise the electrodes as separable components to the microprocessor. For instance, the electrodes may be disposed on an interior surface of the testing chamber, and may extend through a wall of the testing chamber. At an exterior surface of the testing chamber, the electrodes may terminate in electrical contacts. The microprocessor may be in electrical communication with the electrical contacts. In such an embodiment, the only portions of the device in contact with the fluid may be the sample chamber and the testing chamber, as well as the ports/tubes disposed therein for fluidic communication. Thus, the microprocessor may be re-used between multiple patients, while requiring only the sample chamber and testing chamber to be replaced. For instance, the sample chamber, testing chamber and ports/tubes may be provided as a cartridge separate to the rest of the device, and may be fit into the frame. The frame therefore may, in embodiments, comprises fitment features that can create a secure fit with the cartridge.

The electrochemical sensor may further comprise a liquid contact sensor. The liquid contact sensor may be disposed in the interior of the testing chamber at a position relative to the electrodes to ensure that the electrodes are covered by the fluid. The liquid contact sensor may be part of an integrated electrochemical sensor, or use a contact system similar to the electrodes.

The device may further comprise a power supply. The power supply may serve to provide power to the microprocessor. The power supply is not particularly limited, but includes batteries and AC-DC converters. In embodiments of the present disclosure, the device may comprise a fluid sensor. Generally, the fluid sensor may be disposed in a position such that it can detect the inflow of fluid from the inlet port. For instance, the fluid sensor may be disposed in an interior of the inlet port or an interior of the sample chamber.

The device may further comprise a status indicator on an exterior of the device. The status indicator may be in electronic communication with the microprocessor. Upon receiving a signal, the status indicator may produce an audio-visual stimulus, such as an audible sound, an activation of lights, or a combination thereof. For example, if a fluid sample contains a very high concentration of a biomarker, the status indicator may power a red LED, whereas a nominally elevated concentration may cause the powering of a yellow LED, and a healthy concentration may cause the powering of a green LED.

The device may further comprise other sensors. For example, the device may comprise sensors that can measure the volume of fluid passed per hour, the turbidity of the fluid, the temperature of the fluid, urine creatinine content, color index, pH, urea content, salt content, total dissolved solids (TDS), etc.

The material of the device is not particularly limited, but includes polymers, such as a medical grade polymer. In embodiments of the present disclosure, the inlet port, sample in-flow tube and discharge port may comprise a polymer. The polymer of the inlet port, sample in-flow tube and discharge port may be substantially the same as the polymer of the frame. In embodiments, the polymer of the inlet port, sample in-flow tube and discharge port may be a deformable polymer that can allow the use of pinch valves as mentioned previously. The use of a deformable polymer for the port/tubes may allow for valves, such as pinch valves, to be re-used between patients.

The embodiments of the device described herein may be further understood with reference to the figures.

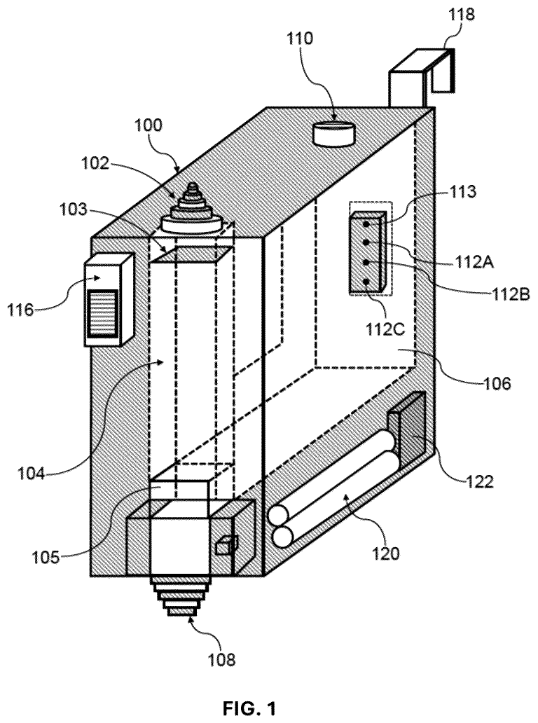

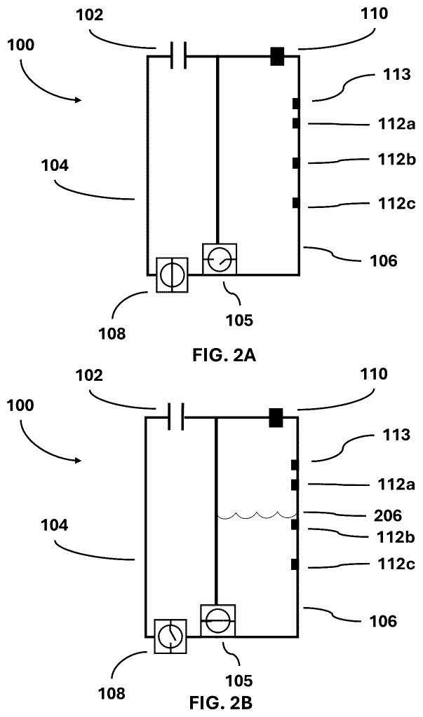

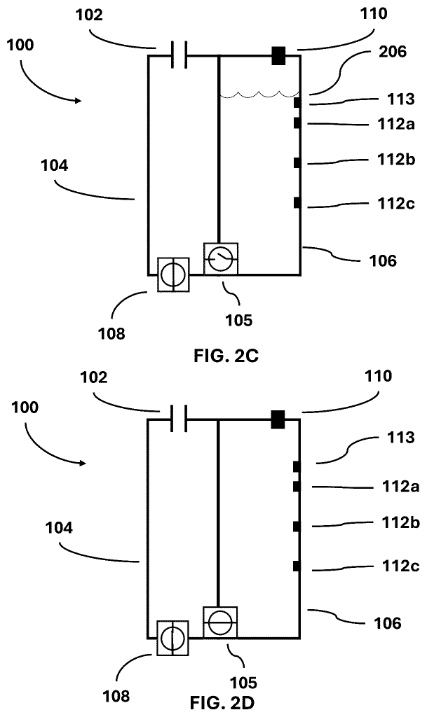

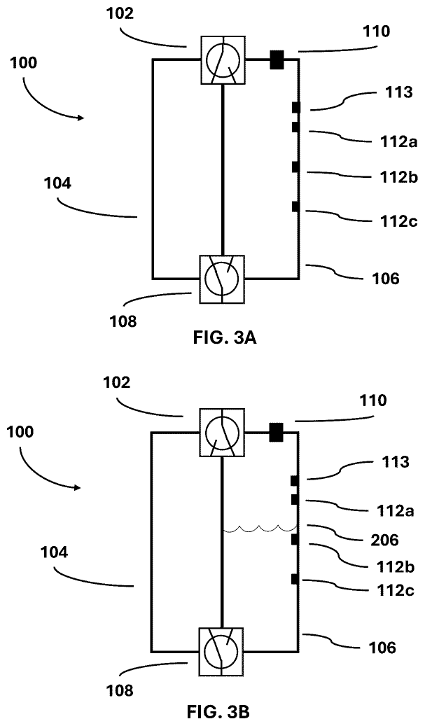

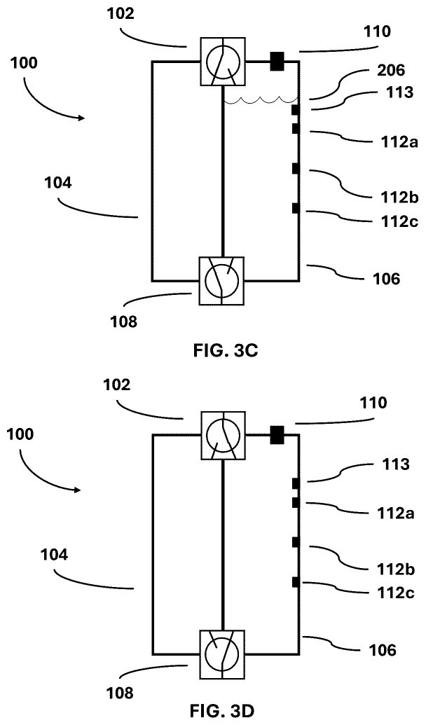

A device 100 is shown in FIG. 1. The device comprises an inlet port 102 defining an aperture between an exterior of the device and the sample chamber 104. Between the inlet port 102 and the sample chamber 104 may be a filter 103. In the sample chamber 104, a sample in-flow tube 105 may define an aperture between the sample chamber 104 and the testing chamber 106. Additionally, the sample chamber 104 may comprise a discharge port 108. The discharge port 108 may allow fluid to exit the device 100. The testing chamber may further comprise a vent 110. As described previously, the vent 110 allows for pressure to equalize in the testing chamber 106 when fluid is drained from the testing chamber 106. Disposed in an interior of the testing chamber 106 are electrodes 112, including working electrode 112a, reference electrode 112b and counter electrode 112c. Liquid contact sensor 113 may be disposed on an interior surface of the testing chamber. Further, the device 100 includes a power supply, shown in FIG. 1 as a bank of batteries 120. The power supply may provide power to the microprocessor 122. The microprocessor 122 may be electrical communication with the electrodes, and the status indicator 116. The device may be positioned using the mounting bracket 118. The device may further comprise a status indicator 116. Details of the device 100 shown in FIG. 1 may be seen in further detail in FIGS. 2A-3D.

A simplified drawing the device 100 of FIG. 1 is shown in FIG. 2A. The device 100 of FIG. 2A comprises the inlet port 102, sample chamber 104, testing chamber 106, discharge port 108, sample in-flow tube 105, electrodes 112, including working electrode 112a, reference electrode 112b and counter electrode 112c, liquid contact sensor 113 and vent 110.

Turning now to FIG. 2B-2C, the operation of the device 100 may be understood. As shown in FIG. 2B, the device 100 may be operated by first closing the discharge port 108 and opening the sample in-flow tube 105. The testing chamber 106 gradually becomes filled with fluid 206 until the fluid level is at or above the liquid contact sensor 113. If the fluid level is above the liquid contact sensor 113 as shown in FIG. 2C, the sample in-flow tube 105 may be closed and the discharge port 108 may be opened. Analysis may then be performed on the fluid 206 by way of the electrodes 112. After analysis has been completed, the sample in-flow tube 105 may be opened as in FIG. 2D to allow the fluid 206 to exit the device 100 through the discharge port 108.

As described previously, the device may comprise 3/2 valves. As shown by FIG. 3A, the inlet port 102 and discharge port 108 comprises a 3/2 valve. Operation of the device 100 is similar as in FIGS. 2A-2D. As shown by FIG. 3B, the testing chamber 106 may be filled with fluid 206 by switching the inlet port 102 to the testing chamber 106, and closing the discharge port 108 to the testing chamber (discharge port 108 is opened to the sample chamber 104). The fluid 206 may fill the testing chamber 106 until the fluid level is at or above the liquid contact sensor 113, as shown by FIG. 3C. Thereafter, analysis on the fluid 206 may be performed using electrodes 112. Finally, the fluid 206 may be drained from the testing chamber 106 by opening the discharge port 108 to the testing chamber 106 (the inlet port 102 may be open to either the testing chamber 106 or the sample chamber 104 as the vent 110 will allow pressure to equalize in the testing chamber 106 as the fluid 206 is drained).

In the embodiments shown within FIGS. 2A-3D, a batch mode is shown. Specifically, the device 100 is operated to take distinct aliquots of fluid 206 for testing. However, it is additionally within the scope of the present disclosure that the device 100 may be operated in a continuous mode. For instance, the discharge port 108 of FIG. 3B may be partially opened to the testing chamber 106, allowing for the slow accumulation (and simultaneous draining) of fluid 206 entering the testing chamber 106. Thereby, the device may continuously measure the concentration of a biomarker within the fluid 206.

The electrodes present within the device may be used to detect the presence and concentration of at least one biomarker, such as through electrochemical techniques, such as cyclic voltammetry and amperometry.

The cyclic voltammetry may be conducted on the fluid using the electrodes, and may be controlled with the microprocessor. The cyclic voltammetry may be performed as is known in the art, i.e., by linearly sweeping the potential between the working electrode and the reference electrode. The current, which is related to the concentration of the biomarker, may be measured between the working electrode and the counter electrode. In an amperometry configuration, a constant potential is applied between a working electrode and a reference electrode, and the resulting current is measured. The magnitude of the measured current is proportional to the concentration of the target analyte in the sample. The data may then be passed to the microprocessor for further processing, an external network or external device where it may be processed. The processing may involve mapping the cyclic voltammetry or amperometry data to a calibration curve, whereby the concentration of the biomarker may be found. The microprocessor may compare the concentration of the biomarker with a reference or threshold value. Examples of threshold values are shown below in Table 1. However, it will be understood that the threshold values shown below are merely exemplary, and that a practitioner of the present invention may decide to use an alternate threshold value. For instance, threshold values may vary according to operative status (pre-operative, peri-operative or post-operative), and patient status (healthy, cardiac stress, cancer, etc.).

TABLE 1
Example thresholds for various biomarkers

| Biomarker | Specimen | Example Threshold | |
|---|---|---|---|
| [TIMP-2]•[IGFB | Urine | 0.3 | ng/mL |
| P7] | | | |
| NGAL | Urine | 100 | ng/mL |
| KIM-1 | Urine (Cr-normalized) | 2.0 | ng/mg Cr |
| IL-18 | Urine | 100 | pg/mL |
| L-FABP | Urine | 28.45 | ng/mL |
| Cystatin C | Serum | 1.055 | mg/L |
| NAG | Urine (Cr-normalized) | 31.65 | μg/g Cr |
| GST (π-GST) | Urine | 14.7 | μg/L |
| Calprotectin | Urine | 300 | ng/mL |
| β2-microglobulin | Urine | 300 | μg/L |
| | Serum | 2.0 | mg/L |
| TFF3 | Urine | 2910 | ng/mL |
| Vanin-1 | Urine (Cr-normalized) | 3.17 | ng/mg Cr |
| Osteopontin | Serum | 577 | ng/mL |

(OPN)

The concentration of the biomarker/risk of AKI may be uploaded to a patient's medical records. The concentration may be further processed through a machine learning model, such as a decoder network, deconvolution network, a model that can interpolate between data points, a model that can extrapolate beyond datapoints, a noise reduction model or a baseline adjustment model. Processing the concentration of the biomarker may allow for a model to assess an AKI status of the patient, or predict a future AKI status of the patient. For example, a plurality of concentrations may be measured over time, wherein the plurality of concentrations may be used to form a trend. The trend may then be compared to the threshold value. In embodiments, the patient specific status, as described previously, may be used to adjust a threshold value.

The device may further possess wireless capabilities. For instance, the device may wirelessly transmit a signal, such as the raw cyclic voltammetry data or the processed cyclic voltammetry data, to an external processor. The method of transmission is not particularly limited, but may include Bluetooth, WiFi, Cellular and RFID. Alternatively, or in combination, the device may possess wire-based communication capabilities. For instance, the device may transmit the signal via Ethernet or USB.

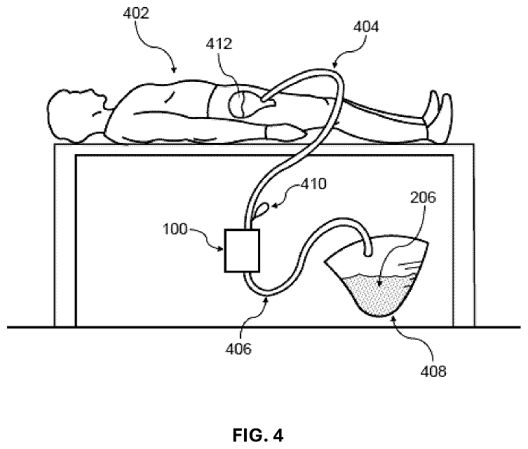

FIG. 4 shows the device 100 connected to a patient 402 through a catheter 404. The catheter may comprise a sampling port 410. A drainage/extension tubing 406 may be coupled to the discharge port of the device 100. The drainage/extension tubing 406 may transmit fluid 206 from the device 100 to a urine collection bag 408. Fluid 206 may initially exit the patient's 402 bladder 412 through the catheter 404. The fluid 206 may travel through the catheter 404 and enter the device 100. The analysis described above may take place, whereinafter the fluid 206 may exit the device 100 through the drainage/extension tubing 406 and enter the urine collection bag 408.

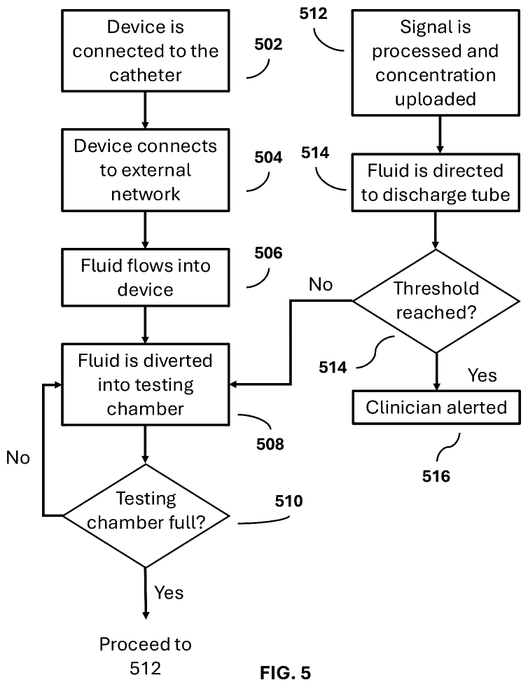

Shown in FIG. 5 is a flowchart of how AKI may be detected in a patient using the device. At 502, the device is connected to the catheter that is inserted into the patient's bladder. At 504, the device may make a connection with an external network. At 506, fluid may flow through the catheter, into the device. At 508, the microprocessor may divert the fluid to the testing chamber for analysis. The microprocessor may loop from step 510 to 508 until the fluid has filled the testing chamber. Thereafter, in step 512, cyclic voltammetry may be performed on the fluid in the testing chamber. The data received from the electrodes in step 512 is processed at step 514, and a concentration/risk of AKI status is uploaded to the patient's medical records. At 514, the output of the microprocessor is processed, and a determination of whether the biomarker concentration has exceeded the threshold is made. If the threshold has not been exceeded, the method proceeds to step 508. If the threshold has been exceeded, clinicians may be alerted at step 516.

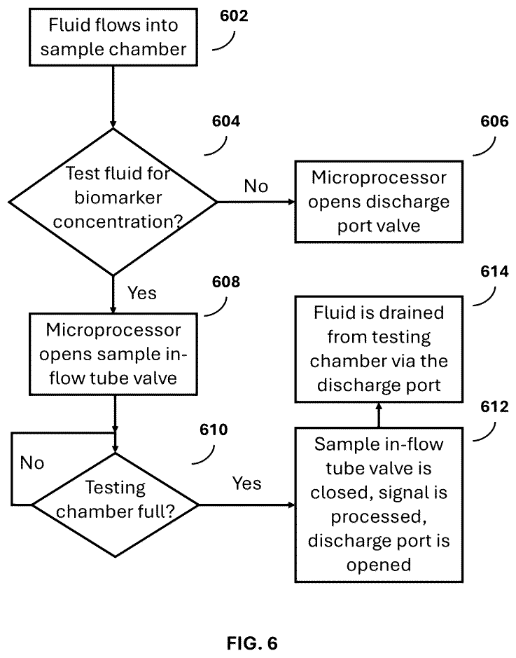

FIG. 6 is a flowchart showing an example of a valve-control sequence used in operating the device. Should the device comprise valves, said valves may be controlled with the microprocessor. A non-transitory computer readable storage medium may contain execution instructions for the valve-control sequence. Starting at step 602, fluid may enter the sample chamber of the device. At step 604, a decision may be made to test the fluid for a concentration of a biomarker, or to pass the fluid through the device. In embodiments of the present disclosure, the non-transitory computer readable storage medium may contain instructions to test fluid for biomarker concentration upon regular intervals, such as every two hours. In embodiments of the present disclosure, the device may be configured to receive a wireless transmission that stimulates the device to test fluid for biomarker concentration. In embodiments of the present disclosure, the device may comprise a user interface, wherein a clinician may input a schedule for testing fluid for biomarker concentration. In embodiments of the present disclosure, the fluid sensor may provide a stimulus to the microprocessor, whereinafter the decision at step 604 may be made. Should the microprocessor's instructions not indicate that a test should be performed, the valve-control sequence may proceed to step 606. At step 606, the discharge port may be opened, such as with a valve, allowing fluid to pass through the device from the inlet port to the discharge port. Should the microprocessor's instructions indicate that a test should be performed, the valve-control sequence may proceed to step 608. At step 608, the microprocessor may open the sample in-flow tube, such as with a valve. In embodiments of the present disclosure, step 608 may further comprise closing the discharge port. At step 610, whether the testing chamber is full is evaluated by the microprocessor, such as by way of the liquid contact sensor. If the testing chamber is not full, the valve states do not change, and the fluid continues to fill the testing chamber. Should the testing chamber be full as determined by the microprocessor by way of the liquid contact sensor, the method proceeds to step 612. At step 612, the sample in-flow tube is closed, the signal from the electrodes is processed by the microprocessor, and the discharge port is opened. After the signal has been processed at step 612, the fluid is drained from the testing chamber via the discharge port, as shown in step 614.

In general, the valve-control sequence may allow for the testing of fluid that flows into the device. Furthermore, the valve-control sequence may prevent fluid from becoming backed up through the inlet port. For example, during operation of the device in batch mode, fluid entering the device by way of the inlet port may be directed to the discharge port while testing is underway in the testing chamber. In this way, fluid does not build up within the device or the catheter.

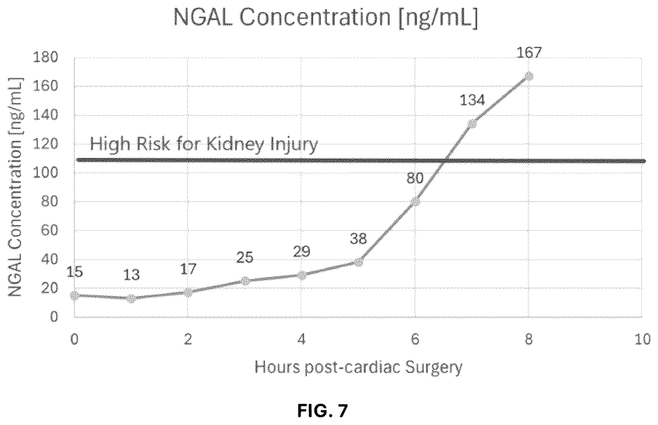

FIG. 7 is graph showing the increase in NGAL concentration as a function of time post-cardiac surgery. As can be seen from the graph, the concentration of NGAL exceeds the critical threshold (in this example) of greater than 110 ng/mL.

In the case of a patient experiencing an increase of NGAL as shown in FIG. 7, the device would add the concentration of NGAL in the patient's medical file, and alert a clinician to the potential AKI status of the patient.

Furthermore, as can be seen in the datapoints prior to the concentration of NGAL surpassing the threshold, the concentration of NGAL steadily increases. Thus, the microprocessor, or other processor as described above, may alert a clinician that a patient is at risk of developing AKI.

What is claimed is:

1. A device for detecting an analyte, the device comprising:
a sample chamber comprising an inlet port, a testing chamber and an electrochemical sensor, wherein the electrochemical sensor is in electrical communication with a working electrode that specifically binds at least one biomarker, and wherein upon binding an output of the working electrode is changed.

2. The device of claim 1, wherein the at least one biomarker is selected from the group consisting of neutrophil gelatinase-associated lipocalin, kidney injury molecule-1 and insulin-like growth factor binding protein 7.

3. The device of claim 1, wherein the sample chamber further comprises a sample in-flow tube and a discharge port, wherein the sample in-flow tube is fluidically connected to a testing chamber.

4. The device of claim 3, wherein the working electrode is disposed on an interior surface of the testing chamber and extends to an exterior surface of the testing chamber.

5. The device of claim 3, wherein the device further comprises solenoid valves in contact with the sample in-flow tube and discharge port.

6. The device of claim 5, wherein the electrochemical sensor further comprises a microprocessor, wherein the microprocessor opens and closes the valves according to a valve-control sequence.

7. The device of claim 1, wherein the working electrode is a molecularly imprinted sensor, an electrochemical aptamer sensor or an electrochemical antibody sensor.

8. The device of claim 1, wherein the electrochemical sensor further comprises a reference electrode and a counter electrode.

9. The device of claim 1, wherein the device wirelessly transmits a signal to an external device.

10. A system comprising a device and a catheter detachably coupled to the device, wherein the device comprises:
a sample chamber comprising an inlet port, a testing chamber and an electrochemical sensor, wherein the electrochemical sensor is in electrical communication with a working electrode that specifically binds at least one biomarker, and wherein upon binding an output of the working electrode is changed.

11. A method for detecting kidney injury in a patient, the method comprising:
collecting a urine sample comprising a biomarker from the patient, wherein the urine is collected through a catheter in fluidic communication with a device, wherein the device comprises:
a sample chamber fluidically connected to the catheter;
a testing chamber fluidically connected to the sample chamber; and
an electrochemical sensor comprising a microprocessor in electrical communication with a working electrode on an interior surface of the testing chamber; and
passing the urine from the catheter to the testing chamber; and
determining a concentration of the biomarker in the urine.

12. The method of claim 11, wherein determining the concentration of the biomarker in the urine is performed with cyclic voltammetry.

13. The method of claim 11, further comprising wirelessly transmitting a signal to an external device.

14. The method of claim 13, wherein the signal is processed by a machine learning model.

15. The method of claim 11, wherein the working electrode is a molecularly imprinted sensor, an electrochemical aptamer sensor or an electrochemical antibody sensor.

16. The method of claim 11, wherein the electrochemical sensor further comprises a reference electrode and a counter electrode.

17. The method of claim 11, wherein the catheter and the device are detachable.

18. The method of claim 11, wherein the device is operated in a continuous flow mode or a batch mode.

19. The method of claim 11, wherein the concentration of the biomarker is compared to a threshold concentration.

20. The method of claim 11, wherein a plurality of concentrations are measured over time, and wherein a trend of the plurality of the concentrations is compared to a threshold concentration.
